SEQUENCE LISTING (50 sequences)

SEQ ID NO: 1 - DNA, 23 bp, Artificial Sequence
  Description of Combined DNA/RNA Molecule designed
  Description of Artificial Sequence designed oligonucleotide
ggaccaccgcaucucuacadtdt

SEQ ID NO: 2 - DNA, 23 bp, Artificial Sequence
  Description of Combined DNA/RNA Molecule designed
  Description of Artificial Sequence designed oligonucleotide
uguagagaugcggugguccdtdt

SEQ ID NO: 3 - DNA, 23 bp, Artificial Sequence
  Description of Artificial Sequence designed oligonucleotide
agcauucguccgguugcgcdtdt

SEQ ID NO: 4 - DNA, 23 bp, Artificial Sequence
  Description of Artificial Sequence designed oligonucleotide
gcgcaaccggacgaaugcudtdt

SEQ ID NO: 5 - DNA, 37 bp, Artificial Sequence
  Description of Artificial Sequence designed oligonucleotide
gaggtggcggcggcggcatgggtgccccgacgttgcc

SEQ ID NO: 6 - DNA, 36 bp, Artificial Sequence
  Description of Artificial Sequence designed oligonucleotide
cccactgagaacgagccagacttggcccagtgtttc

SEQ ID NO: 7 - DNA, 45 bp, Artificial Sequence
  Description of Artificial Sequence designed oligonucleotide
gacgaccccatagaggaacataaaaagcattcgtccggttgcgct

SEQ ID NO: 8 - DNA, 34 bp, Artificial Sequence
  Description of Artificial Sequence designed oligonucleotide
cccagagtggctgcaccacttccagggtttattc

SEQ ID NO: 9 - DNA, 33 bp, Artificial Sequence
  Description of Artificial Sequence designed oligonucleotide
cctgttttgtcttgaaagtggcaccagaggtgc

SEQ ID NO: 10 - DNA, 47 bp, Artificial Sequence
  Description of Artificial Sequence designed oligonucleotide
tttttgggggctcatttttgctgttttgattcccgggcttaccaggt

SEQ ID NO: 11 - DNA, 47 bp, Artificial Sequence
  Description of Artificial Sequence designed oligonucleotide
ttcacagaatagcacaaactacaattaaaactaagcacaaagccatt

SEQ ID NO: 12 - DNA, 30 bp, Artificial Sequence
  Description of Artificial Sequence designed oligonucleotide
cgtctggcagatactccttttgccactgct

SEQ ID NO: 13 - DNA, 48 bp, Artificial Sequence
  Description of Artificial Sequence designed oligonucleotide
aaaggcagtggcctaaatccctttttaaatgacttggctcgatgctgt

SEQ ID NO: 14 - DNA, 33 bp, Artificial Sequence
  Description of Artificial Sequence designed oligonucleotide
cgcagtccgcccaggtccccgctttctttggag

SEQ ID NO: 15 - DNA, 20 bp, Artificial Sequence
  Description of Artificial Sequence designed oligonucleotide
catgccgccgccgccacctc

SEQ ID NO: 16 - DNA, 20 bp, Artificial Sequence
  Description of Artificial Sequence designed oligonucleotide
ggggcacccatgccgccgcc

SEQ ID NO: 17 - DNA, 20 bp, Artificial Sequence
  Description of Artificial Sequence designed oligonucleotide
gcaacgtcggggcacccatg

SEQ ID NO: 18 - DNA, 20 bp, Artificial Sequence
  Description of Artificial Sequence designed oligonucleotide
cccagccttccagctccttg

SEQ ID NO: 19 - DNA, 20 bp, Artificial Sequence
  Description of Artificial Sequence designed oligonucleotide
atgttcctctatggggtcgt

SEQ ID NO: 20 - DNA, 20 bp, Artificial Sequence
  Description of Artificial Sequence designed oligonucleotide
tttatgttcctctatggggt

SEQ ID NO: 21 - DNA, 20 bp, Artificial Sequence
  Description of Artificial Sequence designed oligonucleotide
ccggacgaatgctttttatg

SEQ ID NO: 22 - DNA, 20 bp, Artificial Sequence
  Description of Artificial Sequence designed oligonucleotide
gcaaccggacgaatgctttt

SEQ ID NO: 23 - DNA, 20 bp, Artificial Sequence
  Description of Artificial Sequence designed oligonucleotide
aagcgcaaccggacgaatgc

SEQ ID NO: 24 - DNA, 20 bp, Artificial Sequence
  Description of Artificial Sequence designed oligonucleotide
tggtgcagccactctgggac

SEQ ID NO: 25 - DNA, 20 bp, Artificial Sequence
  Description of Artificial Sequence designed oligonucleotide
aagtggtgcagccactctgg

SEQ ID NO: 26 - DNA, 20 bp, Artificial Sequence
  Description of Artificial Sequence designed oligonucleotide
aataaaccctggaagtggtg

SEQ ID NO: 27 - DNA, 20 bp, Artificial Sequence
  Description of Artificial Sequence designed oligonucleotide
gggaataaaccctggaagtg

SEQ ID NO: 28 - DNA, 20 bp, Artificial Sequence
  Description of Artificial Sequence designed oligonucleotide
ggcaccagggaataaaccct

SEQ ID NO: 29 - DNA, 20 bp, Artificial Sequence
  Description of Artificial Sequence designed oligonucleotide
gctggtggcaccagggaata

SEQ ID NO: 30 - DNA, 20 bp, Artificial Sequence
  Description of Artificial Sequence designed oligonucleotide
caaaaatgagcccccaaaaa

SEQ ID NO: 31 - DNA, 20 bp, Artificial Sequence
  Description of Artificial Sequence designed oligonucleotide
cagcaaaaatgagcccccaa

SEQ ID NO: 32 - DNA, 20 bp, Artificial Sequence
  Description of Artificial Sequence designed oligonucleotide
caaaacagcaaaaatgagcc

SEQ ID NO: 33 - DNA, 20 bp, Artificial Sequence
  Description of Artificial Sequence designed oligonucleotide
tggtaagcccgggaatcaaa

SEQ ID NO: 34 - DNA, 20 bp, Artificial Sequence
  Description of Artificial Sequence designed oligonucleotide
acctggtaagcccgggaatc

SEQ ID NO: 35 - DNA, 20 bp, Artificial Sequence
  Description of Artificial Sequence designed oligonucleotide
agtttgtgctattctgtgaa

SEQ ID NO: 36 - DNA, 20 bp, Artificial Sequence
  Description of Artificial Sequence designed oligonucleotide
gtagtttgtgctattctgtg

SEQ ID NO: 37 - DNA, 20 bp, Artificial Sequence
  Description of Artificial Sequence designed oligonucleotide
tgtagtttgtgctattctgt

SEQ ID NO: 38 - DNA, 20 bp, Artificial Sequence
  Description of Artificial Sequence designed oligonucleotide
tggctttgtgcttagtttta

SEQ ID NO: 39 - DNA, 20 bp, Artificial Sequence
  Description of Artificial Sequence designed oligonucleotide
aatggctttgtgcttagttt

SEQ ID NO: 40 - DNA, 20 bp, Artificial Sequence
  Description of Artificial Sequence designed oligonucleotide
ggatttaggccactgccttt

SEQ ID NO: 41 - DNA, 20 bp, Artificial Sequence
  Description of Artificial Sequence designed oligonucleotide
aaggatttaggccactgcct

SEQ ID NO: 42 - DNA, 20 bp, Artificial Sequence
  Description of Artificial Sequence designed oligonucleotide
caagtcatttaaaaaggatt

SEQ ID NO: 43 - DNA, 20 bp, Artificial Sequence
  Description of Artificial Sequence designed oligonucleotide
gcatcgagccaagtcattta

SEQ ID NO: 44 - DNA, 20 bp, Artificial Sequence
  Description of Artificial Sequence designed oligonucleotide
agcatcgagccaagtcattt

SEQ ID NO: 45 - DNA, 18 bp, Artificial Sequence
  Description of Artificial Sequence designed oligonucleotide
taagctgttctatgtgtt

SEQ ID NO: 46 - DNA, 20 bp, Artificial Sequence
  Description of Artificial Sequence designed oligonucleotide
cagtctcagtactgaagctg

SEQ ID NO: 47 - DNA, 20 bp, Artificial Sequence
  Description of Artificial Sequence designed oligonucleotide
cagcttcagtactgagactg

SEQ ID NO: 48 - DNA, 20 bp, Artificial Sequence
  Description of Artificial Sequence designed oligonucleotide
ggacgggtccgcgaggcacg

SEQ ID NO: 49 - DNA, 19 bp, Artificial Sequence
  Description of Artificial Sequence designed oligonucleotide
gcccgcggtgctaatgctc

SEQ ID NO: 50 - DNA, 19 bp, Artificial Sequence
  Description of Artificial Sequence designed oligonucleotide
gcccgcggtgctaatgctc

(19) United States
(12) Patent Application Publication
(10) Pub. No.: US 2007/0032440 A1
(43) Pub. Date: Feb. 8, 2007

(54) Oligonucleotides directed against a survivin gene and use thereof

(75) Inventors: Axel Meye, Dresden (DE); Susanne Fussel, Dresden (DE); Manfred P. Wirth, Dresden (DE); Uta Schmidt-Strassbuger, Neu-Ulm (DE); Bernd Kuppers, Dresden (DE); Helge Taubert, Grossorner (DE); Matthias Kappler, Halle (DE); Matthias Bache, Halle (DE); Frank Bartel, Leipzig (DE); Peter Wurl, Ulm-Eselsberg (DE)

Correspondence Address: NORRIS, MCLAUGHLIN & MARCUS, P.A., 875 THIRD AVE, 18TH FLOOR, NEW YORK, NY, 10022, US

(73) Assignees: Technische Universitat Dresden, Dresden (DE); Martin-Luther-Universitat Halle-Wittenberg, Halle (DE)

(21) Appl. No.: 10/545,093

(22) PCT Filed: Feb. 9, 2004
(86) PCT No.: PCT/DE04/00263

§ 371 Date: Jun. 8, 2006

(30) Foreign Application Priority Data
Feb. 7, 2003 (DE) 103 06 083.9

(51) Int. Cl. A61K 48/00; C07H 21/04

(52) U.S. Cl. 514/44; 536/23.5

(57) ABSTRACT

The present invention relates to oligonucleotides directed against a survivin gene and variants thereof and to the use of said oligonucleotides in the diagnosis, prophylaxis, reduction and follow-up of diseases associated with cell growth, differentiation and/or division, such as tumor diseases.

The present invention relates to oligonucleotides directed against a survivin gene and to the use of said oligonucleotides in the diagnosis, prophylaxis, reduction and follow-up of diseases associated with cell growth, differentiation and/or division, such as tumor diseases.

Survivin, which belongs to the gene family of the inhibitors of apoptosis (IAP), is a recently discovered link (“interface molecule”) between cell cycle progression and apoptosis control. It is a protein 142 amino acids in length with a molecular weight of about 16.5 kDa. The gene is located on the long branch of chromosome 17 (17q25) (Ambrosini et al., 1997; Adida et al. 1998). Survivin is normally expressed during the embryonal and fetal development, but only to an extremely low extent in adult tissues, contributing to tissue homeostasis and differentiation in the former (Adida et al. 1998). Interestingly, survivin is expressed especially during the G2/M phase. There is a discussion that overexpression of survivin in tumor cells would be capable of deactivating the apoptosis checkpoint in the G2/M phase, allowing progression of transformed cells by mitosis (Li et al. 1998). Survivin is overexpressed in numerous malignant tumors such as lung, colon, stomach, mammary, pancreas, prostate, bladder carcinomas, large-cell non-Hodgkin lymphomas, melanomas and in neuroblastomas, playing an essential role in genesis and particularly in progression of the above tumors (for a review see Altieri et al. 1999). In particular tumors it has been possible to demonstrate correlations between survivin expression and course of a disease or prognosis. Thus, in neuroblastomas, survivin expression correlates with an increasingly disseminating disease (Adida et al. 1998). In colorectal tumors the immunohistochemical detection of survivin protein expression in tumor cells is associated with a reduced rate of apoptosis and a reduced 5-year survival (Kawasaki et al. 1998). In bladder carcinoma, grade 1 tumors with non-detectable survivin protein expression have a relapse-free interval of 35.5 months, while tumors with detectable survivin protein remain free of relapse for only 10.5 months, that is, survivin expression has a predictive value as to the occurrence of relapses in bladder carcinoma (Swana et al. 1999). In the event of glioblastoma, survivin expression is associated with a reduced capability of apoptosis and a significantly worse prospect of survival of the patients (Chakravarti et al. 2002). Furthermore, it has been demonstrated that increased survivin mRNA expression is associated with a worse prognosis for soft-tissue sarcoma (STS) patients (Kappler et al. 2001; Wurl et al. 2002).

Initial tests using survivin antisense oligonucleotides (AS-ON) inducing apoptosis in various tumor cell lines have been highly interesting (Chen et al. 2000; Olie et al. 2000). In addition, it has been shown that transfection of various survivin splice variants in HepG2 cells has an influence on cell survival or apoptosis induction following methotrexate administration (Mahotka et al. 1999). Another study has documented that success in treatment or response to chemotherapy is improved in esophagus cancer patients with low survivin mRNA expression compared to patients with high expression (Kato et al. 2001). Furthermore, it has been demonstrated in the cell line A549 that survivin AS-ON can induce apoptosis or increased sensitivity to the chemotherapeutic agent etoposide (Olie et al. 2000). Likewise, apoptosis is induced by the chemotherapeutic agent cisplatin in various melanoma cell lines, provided they have the phosphorylation-defect survivin mutant (Thr 34 to Ala) (Grossmann et al. 2001). These investigations show that survivin is capable of influencing the induction of apoptosis or the sensitivity to various chemotherapeutic agents. Furthermore, it is well-known that particular antisense constructs are capable of inhibiting the survivin expression (WO 01/057059 A1, U.S. Pat. No. 6,335,194 B1).

Initial results in the prior art have shown a reciprocal correlation between increased survivin mRNA expression and sensitivity to radiation in pancreas carcinoma cell lines, that is, cell lines with elevated survivin mRNA content are more resistant to radiation (Asanuma et al. 2000). Furthermore, latest investigations suggest that survivin is negatively regulated by the tumor suppressor p53 and is involved in p53-dependent apoptosis (Mirza et al. 2002).

Previous disclosures involve the disadvantage that a person skilled in the art has no effective and concrete therapeutical approaches available that could be used in organisms, especially higher organisms such as mammals, particularly humans. A large number of previous disclosures describe constructs very large in size that interact with the survivin gene. When using such large constructs, some effects can be achieved in particular cell suspensions or other cultures. In higher organisms, however, which have an effective immune defense and numerous enzymatic regulatory mechanisms, such large constructs or substances interacting with survivin are subject to immunological attack, destruction or adverse modification before even having a chance of interacting with the actual target molecule to develop a specific effect in the organism. In addition, the stability of relatively large constructs or substances is limited when used in humans. Moreover, the accessibility and target-specific transport of the constructs and substances is limited in the body. Such negative side effects are unpredictable, difficult to control, and—as part of a treatment in humans, e.g. against a malignant tumor disease—therefore represent a relatively high safety risk.

Another drawback is that well-known molecules allow combination with other molecules for chemotherapy, radiotherapy or taxol treatment or the like to only a very limited extent. Furthermore, well-known molecules frequently cannot be used on resistant tumors.

The object of the invention was therefore to provide alternative molecules that would undergo facile, reliable and effective interaction with specific secondary structure motifs of the survivin gene mRNA.

The invention solves the above technical problem by providing oligonucleotides directed towards the mRNA of the survivin gene (data base entry: NM_{—}001168) and/or gene and transcript variants thereof, said oligonucleotides undergoing specific interaction with mRNA target motifs of the survivin gene in a sequence region of from 30 to 1350. The numbers given in the text below refer to the corresponding nucleotide positions within the survivin mRNA (overall length: 1619 nucleotides).

Hence, the invention relates to the unexpected teaching that highly specific and efficient interaction with survivin mRNA is possible with the oligonucleotides according to the invention. The disclosure of the teaching according to the invention enables a person skilled in the art to generate specific oligonucleotides, such as antisense constructs, ribozymes, DNAzymes or siRNA constructs, which interact with the target sequence region in such a way that survivin expression is inhibited, reduced and/or prevented. Apart from these oligonucleotides it is, of course, also possible to select other molecules capable of interacting with the corresponding sequence regions, such as antibodies, Affiline, lectins or aptamers.

The oligonucleotides of the invention can be used in vivo and in vitro e.g. to effect a temporary intracellular attack on the target mRNA of survivin in a specific and efficient manner, thereby inhibiting the oncogenic function of tumor-associated abnormal survivin expression. Thus, the oligonucleotides according to the invention can be used in a diagnostic or therapeutic procedure or in a kit for therapeutical purposes. Another procedure could be, for example, an additive therapy for humans in order to effect local and/or systemic treatment of human tumors, e.g. with other nucleic acid-based constructs, immunotherapeutic agents, chemotherapeutic agents, irradiation and other procedures for tumor treatment in combination with the use of oligonucleotides according to the invention.

Surprisingly, it was shown that oligonucleotides specifically interacting with a sequence region selected from the group comprising the target sequence regions 33-52, 41-60, 49-68, 92-114, 261-280, 264-283, 278-297, 282-301, 283-305, 286-305, 501-520, 504-523, 516-535, 519-538, 526-545, 532-551, 716-735, 719-738, 724-743, 740-759, 743-762, 1126-1145, 1128-1147, 1302-1321, 1304-1323, 1317-1336, 1325-1344 and/or 1327-1346 effectively inhibit the survivin gene, especially by specific interaction of the oligonucleotides with the secondary structure of the mRNA.

It was also surprising that a selection of target sequence regions selected from the group comprising the target sequence regions 27-37, 38-58, 59-79, 93-102, 146-166, 167-187, 173-193, 212-225, 269-279, 280-300, 285-305, 341-361, 381-392, 456-476, 477-497, 490-510, 529-541, 560-577, 566-586, 668-681, 718-738, 737-757,795-807, 843-866, 885-900, 919-939, 840-960, 961-981, 982-1002, 995-1015, 1035-1053, 1072-1098, 1117-1136, 1155-1177, 1196-1215, 1234-1254, 1255-1275, 1294-1314, 1215-1335, 1336-1356, 1352-1372, 1391-1404, 1423-1443, 1444-1464, 1458-1478, 1497-1513, 1532-1552, 1550-1570, 1588-1608 and/or 1599-1619 result in particularly good inhibition of survivin gene expression, the oligonucleotides undergoing particularly effective interaction with specific secondary structure motifs of the survivin gene mRNA.

In a preferred fashion the oligonucleotides interact with the target sequence regions 92-114, 261-280, 264-283, 283-305, 286-305, 504-523, 532-551 and/or 743-762 of the survivin gene mRNA.

Furthermore, in a particularly preferred fashion the siRNAs interact with the regions 92-114, 262-270-IVS2 1-14, 262-270/389-401, 283-305.

In another distinctive embodiment of the invention the sequence region and/or the oligonucleotide is modified by addition, amplification, inversion, missense mutation, nonsense mutation, point mutation, deletion and/or substitution.

It is also preferred to use portions of the above-mentioned target sequences with changes inside or with changed peripheral regions or various derivatizations/modifications/ fusions/complex formations.

In the oligonucleotides, for example, the above modifications and changes in sequence may result in binding to the target with higher specificity. Obviously, however, it may also be envisaged that the oligonucleotides bind with lower specificity.

It is also preferred to combine or fuse the oligonucleotides of the invention with molecules supporting the directed transport to the target site, uptake in and/or distribution inside a target cell; such molecules are well-known to those skilled in the art and have been disclosed in Kappler et al. (2004).

In the meaning of the invention, the mutations in the sequence region of the survivin gene can be heritable or non-heritable changes, for example. For example, the mutations may also include mutations in connection with gene and/or chromosome mutations associated with changes of the survivin gene and transcript variants thereof. Such gene alterations may originate in such a way that portions of the chromosome are lost, doubled, present in reversed orientation, or transferred on other chromosomes. Of course, such a mutation may involve only one or a few adjacent base pairs, as is the case in a point mutation, for example. If, for example, a base pair is lost in the form of a deletion, or if a base pair is interposed in addition, as in insertion, the reading frame of the respective gene will be shifted, resulting in changed triplet coding or premature chain termination (coding of a new stop codon). For example, in a substitution mutation in the meaning of the invention, one base is replaced by another, in which case the resulting consequences may be different:
(a) one codon may be converted into a synonymous codon, for example; or
(b) the mutation changes the codon specificity, resulting in incorporation of other amino acids; or
(c) the mutation causes termination of translation in a particular position, in which case the survivin fragments being formed can be either inactive or active.

In another preferred embodiment the oligonucleotide is a nucleic acid construct. Nucleic acid constructs in the meaning of the invention can be all structures based essentially on nucleic acids, or wherein the active center is based essentially on nucleic acids. Obviously, it is also possible that part of the oligonucleotide/construct consists of lipids, carbohydrates or proteins or peptides, e.g. in the form of a nanocapsule, and that this construct comprises a region containing nucleic acids which, in particular, are capable of interacting with the sequence regions 33-52, 41-60, 49-68, 92-114, 261-280, 264-283, 278-297, 282-301, 283-305, 286-305, 501-520, 504-523, 516-535, 519-538, 526-545, 532-551, 716-735, 719-738, 724-743, 740-759, 743-762, 1126-1145, 1128-1147, 1302-1321, 1304-1323, 1317-1336, 1325-1344 and/or 1327-1346 of the survivin mRNA. Various ways of providing such constructs are well-known to those skilled in the art.

In a preferred embodiment of the invention the oligonucleotide is an antisense oligonucleotide (AS-ON), a DNAzyme, a ribozyme, a peptide nucleic acid (PNA), a so-called locked nucleic acid (LNA) and/or an siRNA.

Accordingly, oligonucleotides (ON), peptide nucleic acids (PNAs), ribozymes or DNAzymes can be used. The AS effect is based on sequence-specific hybridization of the constructs through Watson-Crick base pairing with the target mRNA encoding the protein to be repressed, resulting in reduction/inhibition of protein synthesis via various mechanisms (Table 1).

TABLE 1
AS effects and their mechanisms of action

| Effect | Mechanism | References |
|---|---|---|
| Inhibition of transcription | Binding of the AS constructs to genomic | [Moser et |
| | DNA by Hoogsten triplex formation | al., 1987] |
| Modulation of RNA | a) blocking of splicing sites results in prevention | [Kole et al., 2001; |
| processing | of the splicing process | Crooke 1999] |
| | b) prevention of polyadenylation destabilizes | |
| | the mRNA | |
| | c) obstruction of mRNA transport into the | |
| | cytoplasm | |
| Inhibition of translation | Competitive binding of the AS construct to | [Boiziau et |
| | the target mRNA prevents initiation or elongation | al., 1991] |
| | process | |
| Cleavage of target | a) selective degradation of the RNA strand | [Crooke, 1999; |
| mRNA | in RNA-DNA hybrids by RNase H endonuclease | Agrawal et |
| | b) degradation of ss-RNA by RNase L endonuclease | al., 1998; |
| | after activation by 2′,5′-tetra | Sun et |
| | adenylate-modified ON | al., 2000] |
| | c) ribozyme/DNAzyme-catalyzed, sequence- | |
| | specific cleavage of target | |
| | mRNA | |

ss: single-stranded

Apart from other fields of application, the development of AS-ONs as therapeutic substances also represents a new promising therapeutical concept for oncologic diseases. While conventional chemotherapy results in non-specific inhibition of cell proliferation, the antisense therapy very specifically inactivates those mRNAs which represent the molecular basis or an essential component of degenerate, deregulated growth and tumor progression and may be responsible for the inhibition of the endogenous immune defense.

AS-ONs differ from other therapeutic agents, such as antibodies, toxins or immunotoxins, in that they are relatively small molecules with a molecular weight of normally about 5 kDa. The small size of the AS-ONs allows good tissue penetration. Furthermore, tumor blood vessels, as opposed to blood vessels of normal tissues, are known to be permeable to substances ranging in size between 4 to 10 kDa. Consequently, therapeutic AS-ONs give better penetration of tumor blood vessels. Another advantage of these substances, e.g. with respect to antibodies exclusively effective against extracellular proteins, is that, in addition to membranous proteins, cytoplasmic proteins as well as proteins located in the nucleus can be attacked via the respective target mRNA, using antisense technology.

When using phospothioate oligonucleotides (PS-ONs), so-called “non-antisense” effects advantageously occur in addition to the above-mentioned target-specific AS effects, which, in particular, give rise to non-specific inhibition of cell growth. These effects strongly depend on the oligosequence or on specific sequence motifs, occurring due to strong polyanionic charge of the PS-ONs, which may result in binding of the PS-ONs to vital proteins. The effects mentioned above can be overcome by means of partially phosphothioate-modified AS-ONs or by means of additional modifications, e.g. incorporation of ribonucleotides instead of deoxyribonucleotides. More specifically, terminal modification of ON constructs (preferably 2 to 5 bonds of the 3′ and 5′ nucleic acid terminus) offers increased stability in in vivo applications and in the extra- and intracellular media of target cells, such as, in particular, protection from degradation by exonucleases. One positive effect when using PS-ONs is their immunostimulatory effect which may support possible therapeutic success in some tumor applications.

To increase the stability and specificity of AS-ONs and reduce the “non-AS” effects, further chemical modifications can be employed, e.g. incorporation of 2′-O-methylribonucleotides, methylphosphonate segments, locked nucleic acids (methylene bridge between 2′ oxygen and 4′ carbon of ribose), replacement of cytosine by 5′ -methylcytosine and/or 2′,5′-tetraadenylate modification.

Concerned in this context are partially modified ON constructs or those completely changed via the above chemical modifications.

Being catalytically active RNA molecules, ribozymes are capable of recognizing cellular RNA structures as substrates, cleaving them at a phosphodiester bond of the specific sequence NUX in a sequence-specific fashion. Recognition proceeds via AS branches which, owing to complementary sequences, allow hybridization with the target mRNA. Compared to AS-ONs, ribozymes have the fundamental advantage that a ribozyme molecule, being a true catalyst, is capable of reacting a large number of identical substrate molecules. Consequently, ribozymes are effective at substantially lower concentrations compared to ONs and, in addition, lead to irreversible RNA degradation as a result of substrate cleavage [Sun et al.]. Compared to antisense ONs, ribozymes therefore involve the advantage that a ribozyme molecule, being a true catalyst, is capable of reacting a large number of identical substrate molecules in a multi-turnover reaction. Advantageously, ribozymes are therefore effective at substantially lower concentrations compared to AS-ONs and, in addition, lead to irreversible RNA inhibition as a result of substrate cleavage.

Amongst the types of ribozymes known to date, the hammerhead ribozyme (review: Birikh et al., 1997; Tanner, 1999) is particularly advantageous for such uses because it has catalytic activity even as a comparatively small molecule (about 30-50 nucleotides). For example, a highly effective trans-cleaving hammerhead ribozyme consists of no more than 14 conserved nucleotides in the catalytic domain and two variable ancestral sequences—each advantageously made up of 6 to 8 nucleotides—which, via Watson-Crick base pairing, in analogy to AS-ON, accomplish sequence-specific recognition of the substrate to be cleaved, subsequently inactivating the latter by cleavage of a phosphodiester bond. In this fashion, it is possible with advantage to construct a specifically cleaving hammerhead ribozyme against any RNA molecule having a potential cleavage site with the minimum sequence requirement -NUX- (X=any nucleotide other than G).

“RNA interference” (RNAi) as a methodology of gene inhibition is advantageously mediated by small, synthetically produced RNA oligonucleotides (“small interfering RNAs”: siRNA) allowing selective inhibition of the intracellular survivin synthesis. These siRNAs are specific reagents preferably used to inhibit gene expression in pro- and eukaryotic cells. RNA interference is induced by double-stranded RNA (dsRNA), resulting in sequence-specific cleavage of single-stranded target RNA (mRNA) having appropriate sequence homology to said dsRNA. The actual mediators of mRNA degradation are short interfering dsRNAs (siRNAs) formed by cellular enzyme systems as cleavage products from the long dsRNAs. These short RNA duplexes (siRNAs) have a characteristic length of 21 to 23 nt, with a single-strand overhang 2 nt in length at each 3′ terminus of both chains. Now, the sequence of this siRNA determines the recognition of homologous mRNA regions and their degradation through activation of cellular RNases.

In this way, the protein synthesis of the target gene to be repressed can be directly influenced, thereby inducing elimination of the target protein. Thus, by inhibiting the translation of survivin and variants thereof, the siRNAs block the flow of information in the cell. In addition, siRNAs advantageously are capable of inhibiting other RNA molecules which are not translated and consequently take effect on the RNA level only.

In another preferred embodiment of the invention the oligonucleotide/oligonucleotides is/are immobilized. In the meaning of the invention, immobilization is understood to involve various methods and techniques to fix the oligonucleotides on specific carriers. For example, immobilization can serve to stabilize the oligonucleotides so that their activity would not be reduced or adversely modified by biological, chemical or physical exposure, especially during storage or in single-batch use. Immobilization of the oligonucleotides allows repeated use under technical or clinical routine conditions. Furthermore, a sample can be reacted with the oligonucleotides in a continuous fashion. In particular, this can be achieved by means of various immobilization techniques, with binding of the oligonucleotides to other oligonucleotides or molecules or to a carrier proceeding in such a way that the three-dimensional structure in the active center of the corresponding molecules, especially of said oligonucleotides, would not be changed. Advantageously, there is no loss in specificity to the sequence regions of the target and in specificity of the actual binding reaction as a result of such immobilization. In the meaning of the invention, three basic methods can be used for immobilization:
(i) Crosslinking: in crosslinking, the oligonucleotides are fixed to one another without adversely affecting their activity. Advantageously, they are no longer soluble.
(ii) Binding to a carrier: binding to a carrier proceeds via adsorption, ionic binding or covalent binding, for example. Such binding may also take place inside microbial cells or liposomes or other membranous, closed or open structures. Advantageously, the oligonucleotide is not adversely affected in its specificity/activity by such fixing. For example, carrier-bound multiple or continuous use thereof is possible with advantage in clinics in diagnosis or therapy.
(iii) Inclusion: inclusion in the meaning of the invention especially is inclusion in a semipermeable membrane in the form of gels, fibrils or fibers. Advantageously, encapsulated oligonucleotides are separated from the surrounding sample solution by a semipermeable membrane in such a way that specific interaction with the sequence regions 33-52, 41-60, 49-68, 92-114, 261-280, 264-283, 278-297, 282-301, 283-305, 286-305, 501-520, 504-523, 516-535, 519-538, 526-545, 532-551, 716-735, 719-738, 724-743, 740-759, 743-762, 1126-1145, 1128-1147, 1302-1321, 1304-1323, 1317-1336, 1325-1344 and/or 1327-1346 still is possible.

Various methods are available for immobilization, such as adsorption on an inert or electrically charged inorganic or organic carrier. For example, such carriers can be porous gels, aluminum oxide, bentonite, agarose, starch, nylon or polyacrylamide. Immobilization proceeds via physical binding forces, frequently involving hydrophobic interactions and ionic binding. Advantageously, such methods are easy to handle, having no or only little influence on the conformation of the oligonucleotides. Advantageously, binding can be improved as a result of electrostatic binding forces between the charged groups of the oligonucleotides and the carrier. The surface of microscopic porous glass particles can be activated by treatment with silanes and subsequently reacted with oligonucleotides. Advantageously, a large number of oligonucleotides can undergo direct covalent binding with polyacrylamide resins. Inclusion in three-dimensional networks involves inclusion of the oligonucleotides in ionotropic gels or other structures well-known to those skilled in the art. More specifically, the pores of the matrix are such in nature that the oligonucleotides are retained, allowing interaction with the target molecules or sequence regions 33-52, 41-60, 49-68, 92-114, 261-280, 264-283, 278-297, 282-301, 283-305, 286-305, 501-520, 504-523, 516-535, 519-538, 526-545, 532-551, 716-735, 719-738, 724-743, 740-759, 743-762, 1126-1145, 1128-1147, 1302-1321, 1304-1323, 1317-1336, 1325-1344 and/or 1327-1346. In microencapsulation, the reaction volume of the oligonucleotides is restricted by means of membranes. For example, microencapsulation can be carried out in the form of an interfacial polymerization. Owing to the immobilization during microencapsulation, the oligonucleotides are made insoluble and thus reusable. In the meaning of the invention, immobilized oligonucleotides are all those oligonucleotides being in a condition that allows reuse thereof. Restricting the mobility and solubility of the oligonucleotides by chemical, biological or physical means advantageously results in lower process cost.

The invention also relates to a pharmaceutical composition comprising the oligonucleotides of the invention, optionally in combination with a pharmaceutically tolerable carrier. More specifically, the pharmaceutical carrier may comprise additional materials and substances such as medical and/or pharmaceutical-technical adjuvants. For example, medical adjuvants are materials used as ingredients in the production of pharmaceutical compositions. Pharmaceutical-technical adjuvants serve to suitably formulate the drug or pharmaceutical composition and, if required during the production process only, can even be removed thereafter, or they can be part of the pharmaceutical composition as pharmaceutically tolerable carriers. Formulation of the pharmaceutical composition is optionally effected in combination with a pharmaceutically tolerable diluent. For example, the diluents can be phosphate-buffered saline, water, emulsions such as oil/water emulsions, various types of detergents, sterile solutions, and the like. For example, the pharmaceutical composition can be administered in association with a gene therapy, e.g. via suitable vectors, such as viral vectors. The kind of dosage and route of administration can be determined by the attending physician according to the clinical factors. As is familiar to those skilled in the art, the kind of dosage will depend on various factors, such as size, body surface, age, sex, or general health condition of the patient, but also on the particular agent being administered, the time period and type of administration, and on other medications possibly administered in parallel, especially in a combination therapy.

The invention also relates to a kit comprising said oligonucleotides and/or said pharmaceutical composition. Furthermore, the invention relates to an array comprising the oligonucleotides and/or the pharmaceutical composition. Kit and array can be used in the diagnosis and/or therapy of diseases associated with the activity of the survivin gene.

The invention also relates to the use of said oligonucleotides, said kit, said array in the diagnosis, prophylaxis, reduction, therapy, follow-up and/or aftercare of diseases associated with cell growth, differentiation and/or division, preferably benign and malignant tumor diseases (neoplasms), other hyper- and/or dysproliferative diseases.

In a preferred embodiment the disease associated with cell growth, differentiation and/or division is a tumor. In a particularly preferred fashion the tumor is a solid tumor and/or blood or lymphatic node cancer.

More specifically, the tumors in the meaning of the invention, which can be of epithelial or mesodermal origin, can be benign or malignant types of tumors in organs such as lungs, prostate, urinary bladder, kidneys, esophagus, stomach, pancreas, brain, ovaries, skeletal system, with adenocarcinoma of breast, prostate, lungs and intestine, bone marrow cancer, melanoma, hepatoma, ear-nose-throat tumors in particular being explicitly preferred as members of so-called malignant tumors. In the meaning of the invention, the group of blood or lymphatic node cancer types includes all forms of leukemias (e.g. in connection with B cell leukemia, mixed-cell leukemia, null cell leukemia, T cell leukemia, chronic T cell leukemia, HTLV-II-associated leukemia, acute lymphocytic leukemia, chronic lymphocytic leukemia, mast cell leukemia, and myeloid leukemia) and lymphomas. The following entities are preferred as examples of mesenchymal malignant tumors (so-called bone and soft-tissue sarcomas): fibrosarcoma; malignant histiocytoma; liposarcoma; hemangiosarcoma; chondrosarcoma and osteosarcoma; Ewing sarcoma; leio- and rhabdomyosarcoma, synovialsarcoma; carcinosarcoma. More specifically, further types of tumors, also summarized under the term of “neoplasms”, are the following: bone neoplasms, breast neoplasms, neoplasms of the digestive system, colorectal neoplasms, liver neoplasms, pancreas neoplasms, hypophysis neoplasms, testicle neoplasms, orbital neoplasms, neoplasms of head and throat, of the central nervous system, neoplasms of the hearing organ, pelvis, respiratory tract and urogenital tract.

In another preferred embodiment the cancerous disease or tumor being treated or prevented is selected from the group of: tumors of the ear-nose-throat region, comprising tumors of the inner nose, nasal sinus, nasopharynx, lips, oral cavity, oropharynx, larynx, hypopharynx, ear, salivary glands, and paragangliomas, tumors of the lungs, comprising non-parvicellular bronchial carcinomas, parvicellular bronchial carcinomas, tumors of the mediastinum, tumors of the gastrointestinal tract, comprising tumors of the esophagus, stomach, pancreas, liver, gallbladder and biliary tract, small intestine, colon and rectal carcinomas and anal carcinomas, urogenital tumors comprising tumors of the kidneys, ureter, bladder, prostate gland, urethra, penis and testicles, gynecological tumors comprising tumors of the cervix, vagina, vulva, uterine cancer, malignant trophoblast disease, ovarian carcinoma, tumors of the uterine tube, tumors of the abdominal cavity, mammary carcinomas, tumors of the endocrine organs, comprising tumors of the thyroid, parathyroid, adrenal cortex, endocrine pancreas tumors, carcinoid tumors and carcinoid syndrome, multiple endocrine neoplasias, bone and soft-tissue sarcomas, mesotheliomas, skin tumors, melanomas comprising cutaneous and intraocular melanomas, tumors of the central nervous system, tumors during infancy, comprising retinoblastoma, Wilms tumor, neurofibromatosis, neuroblastoma, Ewing sarcoma tumor family, rhabdomyosarcoma, lymphomas comprising non-Hodgkin lymphomas, cutaneous T cell lymphomas, primary lymphomas of the central nervous system, Hodgkin's disease, leukemias comprising acute leukemias, chronic myeloid and lymphatic leukemias, plasma cell neoplasms, myelodysplasia syndromes, paraneoplastic syndromes, metastases with unknown primary tumor, peritoneal carcinomatosis, immunosuppression-related malignancy comprising AIDS-related malignancies such as Kaposi sarcoma, AIDS-associated lymphomas, AIDS-associated lymphomas of the central nervous system, AIDS-associated Hodgkin disease, and AIDS-associated anogenital tumors, transplantation-related malignancy, metastasized tumors comprising brain metastases, lung metastases, liver metastases, bone metastases, pleural and pericardial metastases, and malignant ascites.

In a distinctive embodiment of the invention the solid tumor is a tumor of the urogenital tract and/or gastrointestinal tract.

In another particularly preferred embodiment of the invention it is envisaged that the tumor is a colon carcinoma, stomach carcinoma, pancreas carcinoma, a colon cancer, small intestine cancer, an ovarian carcinoma, cervical carcinoma, a lung cancer, a renal cell carcinoma, a brain tumor, a head- throat tumor, a liver carcinoma and/or a metastase of the above tumors/carcinomas.

In another particularly preferred embodiment the solid tumor is a mammary, bronchial, colorectal and/or prostate carcinoma.

In a most preferred embodiment the tumor of the urogenital tract is a bladder carcinoma (BCa). In the Federal Republic of Germany, BCa represents the fourth most frequent form of cancer and the seventh most frequent cause of cancer death in males. TUR-B as a general primary therapy of BCa allows organ-preserving removal of superficial tumors. Despite such histopathologically defined complete removal of the tumor, a relatively high percentage of patients, being from 50 to 70%, experience a relapse within two years [Stein et al.]. One problem in diagnosis and therapy is the synchronous or metachronous multifocal appearance of tumor centers, which may be a possible cause of the appearance of relapses remote from the resected primary tumor location [Sidransky et al.]. In cases of appearing relapses or tumors primarily classified as superficial, the TUR-B is normally followed by a long-term prophylaxis using an immunotherapeutic (bacillus Calmette Guérin; BCG) or chemotherapeutic agent (e.g. mitomycin C, taxol, gemcitabin/cisplatin). Patients with muscle-invasive BCa and dedifferentiated superficial tumors, who experience relapse despite such therapy, are normally treated with radical cystectomy or, preserving the bladder, by means of mono-/polychemo-, immuno- or radiotherapy or combined procedures of these methods. Due to their relatively unspecific mechanisms of action, chemical, immune or radiation treatments are accompanied by high therapy-induced toxicity.

Due to the importance of BCa in health policy (especially in Western industrial nations), lack of tumor-specific markers, and well-known tumor-biological and cellular heterogeneity of the tumor, there is an intense search in the field of clinical research on bladder carcinoma, particularly with the aim of identifying new or/and supplementing therapeutical options.

In a distinctive embodiment of the invention the oligonucleotide, the pharmaceutical composition, the kit and/or the array are used in a follow-up essentially representing monitoring the effectiveness of an anti-tumor treatment. Furthermore, it is preferred that the oligonucleotide be used in a combination therapy, especially for the treatment of tumors. In a particularly preferred fashion, said combination therapy comprises a chemotherapy, a treatment with cytostatic agents and/or a radiotherapy.

In a particularly preferred embodiment of the invention the combination therapy is an adjuvant, biologically specific form of therapy, and in a particularly preferred fashion, said form of therapy is an immune therapy. Furthermore, in a particularly preferred fashion the combination therapy comprises a gene therapy and/or a therapy using an oligonucleotide against the same or other target molecule. In the meaning of the invention, gene therapy is a form of treatment using natural or recombinantly engineered nucleic acid constructs, single gene sequences or complete gene or chromosome sections or encoded transcript regions, derivatives/modifications thereof, with the objective of a biologically based and selective inhibition or reversion of disease symptoms and/or the causal origin thereof, in special cases this being understood to involve inhibition of a target molecule on a nucleic acid level, especially transcript level, which has been overexpressed in the course of a disease.

Various combination therapies, especially for the treatment of tumors, are well-known to those skilled in the art. For example, a treatment with cytostatic agents or e.g. irradiation of a particular tumor area can be envisaged within the scope of a combination therapy, and this treatment is combined with a gene therapy, using the oligonucleotide of the invention as an anticancer agent. However, the oligonucleotide according to the invention can also be used in combination with other oligonucleotides directed against the same target molecule or against a completely different structure. Accordingly, in a particularly preferred fashion the oligonucleotide can be used to increase the sensitivity of tumor cells to cytostatic agents and/or radiation. Furthermore, a preferred use of the oligonucleotide is in inhibiting the viability and the proliferation rate of cells and/or inducing apoptosis and cell cycle arrest.

Without intending to be limiting, the invention will be explained in more detail with reference to the examples.

### EXAMPLES

1. Antisense constructs (antisense ON) directed towards survivin and effectiveness thereof

Potential target motifs within the target mRNA of survivin were identified in the course of preliminary investigations, which, being single-stranded structures, should be readily accessible for hybridization. A total of 26 AS-ONs and five nonsense ONs and various antisense oligonucleotides against said single-stranded motifs (ss motifs) were designed and are summarized in Table 2.

We selected the BCa cell line EJ28 as BCa model because lipid-mediated ON transfection succeeded efficiently in preliminary investigations and the cells showed distinct mRNA and survivin protein expression (Kappler et al., 2004). Surprisingly, in direct comparison to the nonsense ON control batches, various AS constructs showed an efficient viability-inhibiting and target-specific antisense effect detectable on various investigation levels. Particularly in viability investigations (STS-1 Test, Roche), marked reduction in 11 out of 31 tested anti-survivin AS-ON constructs (Table 2) could be clearly detected (FIG. 1). Basically, specific inhibition of viability was detectable within a time period of from 12 to 48 hours in an ON concentration range of from 250 to 750 nM following single transfection.

The inhibition of tumor cell viability directly correlated with a detectable reduction of the survivin mRNA expression rate (detection via quantitative Taq-Man RT-PCR, FIG. 2) and with the corresponding protein expression level (detection via Western blot and survivin ELISA, FIGS. 3 and 4), being about one third in the AS-ON-transfected cells when compared to likewise treated nonsense ON control batches.

These findings demonstrate efficient internalization, target hybridization and blocking or degradation of the survivin mRNA target molecules.

TABLE 2

Antisense and nonsense ON constructs
The designation refers to the first position
(in 5′→3′ direction) within the target mRNA
of survivin (data base entry: NM_001168) which
undergoes hybridization with the corresponding
antisense construct.
*The control ON given below was taken from a
published study (Chen et al. 2000).

| | | length |
|---|---|---|
| Designation | Antisense ON sequence (5′→3′) | (nt) |
| SVV 0033 | catgccgccgccgccacctc | 20 |
| SVV 0041 | ggggcacccatgccgccgcc | 20 |
| SVV 0049 | gcaacgtcggggcacccatg | 20 |
| SVV 0261 | atgttcctctatggggtcgt | 20 |
| SVV 0264 | tttatgttcctctatggggt | 20 |
| SVV 0278 | ccggacgaatgctttttatg | 20 |
| SVV 0282 | gcaaccggacgaatgctttt | 20 |
| SVV 0286 | aagcgcaaccggacgaatgc | 20 |
| SVV 0501 | tggtgcagccactctgggac | 20 |
| SVV 0504 | aagtggtgcagccactctgg | 20 |
| SVV 0516 | aataaaccctggaagtggtg | 20 |
| SVV 0519 | gggaataaaccctggaagtg | 20 |
| SVV 0526 | ggcaccagggaataaaccct | 20 |
| SVV 0532 | gctggtggcaccagggaata | 20 |
| SVV 0716 | caaaaatgagcccccaaaaa | 20 |
| SVV 0719 | cagcaaaaatgagcccccaa | 20 |
| SVV 0724 | caaaacagcaaaaatgagcc | 20 |
| SVV 0740 | tggtaagcccgggaatcaaa | 20 |
| SVV 0743 | acctggtaagcccgggaatc | 20 |
| SVV 1126 | tggctttgtgcttagtttta | 20 |
| SVV 1128 | aatggctttgtgcttagttt | 20 |
| SVV 1302 | ggatttaggccactgccttt | 20 |
| SVV 1304 | aaggatttaggccactgcct | 20 |
| SVV 1317 | caagtcatttaaaaaggatt | 20 |
| SVV 1325 | gcatcgagccaagtcattta | 20 |
| SVV 1327 | agcatcgagccaagtcattt | 20 |
| NS-1* | taagctgttctatgtgtt | 18 |
| NS-2 | cagtctcagtactgaagctg | 20 |
| NS-3 | cagcttcagtactgagactg | 20 |
| NS-4 | ggacgggtccgcgaggcacg | 20 |
| NS-5 | gcccgcggtgctaatgctc | 19 |

[image]

[image]

[image]

[image]

2. siRNA Constructs Directed Towards Survivin and Effectiveness Thereof

Five tumor cell lines (sarcoma cell line US 8/93 [Taubert et al. 1997], two rhabdomyosarcoma cell lines (RD: CCL-136 and A204: HTB-82), one leiomyosarcoma cell line SK-LMS: HTB-88 and one osteosarcoma cell line Saos-2: HTB 85) were investigated with respect to their behavior following treatment with siRNA (Table 4) directed against survivin (IAP). The above cell lines are tumor-biologically well-characterized sarcoma cell lines.

TABLE 4

Anti-survivin siRNA constructs and associated
nonsense siRNA control
The numbering of the target motif relates to
the data base entry NM_001168 of the survivin
mRNA (s = sense, as = antisense).

| | | mRNA |
|---|---|---|
| | | target |
| siRNA-ON | Sequence | motif |
| siRNA 1 exon1 s | ggaccaccgcaucucuacadtdt | 92-114 |
| siRNA 2 exon1 as | uguagagaugcggugguccdtdt | 92-114 |
| siRNA 3 nonsense3 | acaucucuacgccaccaggdtdt | — |
| siRNA 4 nonsense4 | ccugguggcguagagaugudtdt | — |
| siRNA 5 exon3 | agcauucguccgguugcgcdtdt | 283-305 |
| siRNA 6 exon3 | gcgcaaccggacgaaugcudtdt | 283-305 |

Following single treatment with siRNA (300 nM) (siRNAs 1 and 2 directed against survivin 92-114, and siRNAs 3 and 4 as nonsense control), the cells thus therapeutically treated showed a) reduced amounts of survivin mRNA and protein, b) increased apoptosis detected using cell supernatant analyses, c) strongly reduced cell survival in a cell colony formation test, and d) appearance of giant cells (polyploid). These analyses were standardized with respect to a comparative group treated under the same conditions, which, unlike the analysis group, was treated with a control RNAi construct having no target gene (nonsense control).

The reduction of the survivin mRNA by specific siRNA is stable for about 3 days, documenting the effect of therapy over time (FIG. 6), the survivin mRNA being reduced by up to 90% compared to the control (FIGS. 5 and 6). In accordance with the initial expression, there are marked differences between different cell lines. A reduction of the survivin mRNA down to 2 zmol survivin/amol GAPDH could be detected in the investigations.

[image]

[image]

Corresponding to the reduction of the mRNA, the product of the survivin mRNA, i.e., the survivin protein, was also reduced by about 50% (FIG. 7).

The essential cell-biological effect of specific survivin inhibition was a significant increase of apoptosis. The latter was determined by estimating the floating cells in the culture flask, and 70-90% of all cells found in the supernatant had become apoptotic (determination of cell nucleus morphology following DAPI staining). A rate of apoptosis progressing with time was found in cells treated with anti-survivin siRNA, and up to 19% of all cells had become apoptotic after 3 days. This reaction could be verified by detecting reduced survival of cells thus treated. Cell colony formation tests showed a reduced cell viability of about 50% compared to the nonsense control (FIG. 8).

[image]

For an illustration of the survivin protein expression following siRNA application, see above.

[image]

Supplementing the surprising effects on tumor cell growth following single treatment with oligonucleotides, the above-mentioned constructs were combined with various chemotherapeutical agents. The BCa-relevant cytostatic agents cisplatin, gemcitabin and mitomycin C were used. The transfections with the above-mentioned oligonucleotides prior to chemotherapy resulted in remarkable and unexpected enhancing effects with respect to the cytostatic effect compared to combination with the control constructs. The combined treatment serves in furnishing specificity of a chemotherapy, thus allowing dosage minimization in conventional forms of therapy. Moreover, in some tumor cell lines regarded as resistant per se, it was possible to obtain sensitization to particular chemotherapeutical agents.

In animal experiments using human tumor cells (nude rat xenograft model), treatment in the form of fractionated instillation of human BCa cells implanted in the bladder gave complete tumor regression of small tumors. In comparison, it was possible to stop the progression of larger, infiltratively growing tumors. Surprisingly, these effects of proliferative inhibition were accompanied by complete regression of blood vessels in larger tumors. Unexpectedly, the survivin-directed and specific form of therapy in vivo was accompanied by spontaneous and complete inhibition of neoangiogenesis over a period of several weeks.

Supplementing these surprising results, a combination of siRNA treatment against survivin with taxol treatment resulted in a sensitization to such taxol treatment.

Likewise surprising was the result that treatment with siRNA against survivin in combination with an irradiation treatment gave a decrease in surviving cell colonies, thus resulting in a radiosensitization in the cell line A204 (FIG. 9). Using irradiation with 4 Gy and siRNA treatment, an enhancement factor of 2.5 over radiotherapy alone was obtained.

[image]

Relevant References:

Agrawal S, Zhao Q: Antisense therapeutics. Curr Opin Chem Biol (1998) 2: 519-28.

Ambrosini G, Adida C, Altieri D C. A novel anti-apoptosis gene, survivin, expressed in cancer and lymphoma. Nat Med. (1997) 3: 917-21.

Asanuma K, Moriai R, Yajima T, Yagihashi A, Yamada M, Kobayashi D, Watanabe N. Survivin as a radioresistance factor in pancreatic cancer. Jpn J Cancer Res. 2000 91: 1204-9.

Boiziau C, Kurfurst R, Cazenave C, Roig V, Thuong N T, Toulme J J: Inhibition of translation initiation by antisense oligonucleotides via an RNase-H independent mechanism. Nucleic Acids Res (1991) 19: 1113-9.

Chen J, Wu W, Tahir S K, Kroeger P E, Rosenberg S H, Cowsert L M, Bennett F, Krajewski S, Krajewska M, Welsh K, Reed J C, Ng S C. Down-regulation of survivin by antisense oligonucleotides increases apoptosis, inhibits cytokinesis and anchorage-independent growth. Neoplasia (2000) 2:23541.

Crooke S T: Molecular mechanisms of action of antisense drugs. Biochim Biophys Acta (1999) 1489: 3144.

Kole R, Sazani P: Antisense effects in the cell nucleus: modification of splicing. Curr Opin Mol Ther (2001) 3: 229-34.

Grossman D, Kim P J, Schechner J S, Altieri D C. Inhibition of melanoma tumor growth in vivo by survivin targeting. Proc Natl Acad Sci USA (2001) 98: 63540.

Kappler M, Kohler T, Kampf C, Diestelkotter P, Wurl P, Schmitz M, Bartel F, Lautenschlager C, Rieber E P, Schmidt H, Bache M, Taubert H, Meye A. Increased survivin transcript levels: an independent negative predictor of survival in soft tissue sarcoma patients. Int J Cancer (2001) 95: 360-3.

Kato J, Kuwabara Y, Mitani M, Shinoda N, Sato A, Toyama T, Mitsui A, Nishiwaki T, Moriyama S, Kudo J, Fujii Y. Expression of survivin in esophageal cancer: correlation with the prognosis and response to chemotherapy. Int J Cancer (2001) 95: 92-5.

Kawasaki H, Altieri D C, Lu C D, Toyoda M, Tenjo T, Tanigawa N. Inhibition of apoptosis by survivin predicts shorter survival rates in colorectal cancer. Cancer Res (1998) 58: 5071-4.

Li F, Ambrosini G, Chu E Y, Plescia J, Tognin S, Marchisio P C, Altieri D C. Control of apoptosis and mitotic spindle checkpoint by survivin. Nature (1998) 396: 580-4.

Mahotka C, Wenzel M, Springer E, Gabbert H E, Gerharz C D. Survivin-deltaEx3 and survivin-2B: two novel splice variants of the apoptosis inhibitor survivin with different antiapoptotic properties. Cancer Res (1999) 59: 6097-102.

Mirza A, McGuirk M, Hockenberry T N, Wu Q, Ashar H, Black S, Wen S F, Wang L, Kirschmeier P, Bishop W R, Nielsen L L, Pickett C B, Liu S. Human survivin is negatively regulated by wild-type p53 and participates in p53-dependent apoptotic pathway. Oncogene (2002) 21: 2613-22.

Moser H E, Dervan P B: Sequence-specific cleavage of double helical DNA by triple helix formation. Science (1987) 238: 645-50.

Olie R A, Simoes-Wust A P, Baumann B, Leech S H, Fabbro D, Stahel R A, Zangemeister-Wittke U. A novel antisense oligonucleotide targeting survivin expression induces apoptosis and sensitizes lung cancer cells to chemotherapy. Cancer Res (2000) 60: 2805-9.

Sun L Q, Cairns M J, Saravolac E G, Baker A, Gerlach W L: Catalytic nucleic acids: from lab to applications. Pharmacol Rev (2000) 52: 32547.

Swana H S, Grossman D, Anthony J N, Weiss R M, Altieri D C. Tumor content of the antiapoptosis molecule survivin and recurrence of bladder cancer. N Engl J Med (1999) 341:452-3.

1. An oligonucleotide, said oligonucleotide being directed against a survivin gene, characterized in that the oligonucleotide undergoes specific interaction with a survivin mRNA in a target sequence region selected from the group comprising the target sequence regions 33-52, 41-60, 49-68, 92-114, 261-280, 264-283, 278-297,282-301,283-385,286-305,501-520,504-523, 516-535,519-538, 526-545, 532-551, 716-735, 719-738, 724-743, 740-759, 743-762, 1126-1145, 1128-1147, 1302-1321, 1304-1323, 1317-1336, 1325-1344 and/or 1327-1346.

2. The oligonucleotide according to claim 1, characterized in that said sequence region and/or said oligonucleotide is modified by addition, amplification, inversion, missense mutation, nonsense mutation, point mutation, deletion and/or substitution.

3. The oligonucleotide according to claim 1, characterized in that the oligonucleotide is immobilized.

4. The oligonucleotide according to claims 1, characterized in that the oligonucleotide is a nucleic acid construct.

5. The oligonucleotide according to claim 4, characterized in that the oligonucleotide is fused or complexed with another molecule supporting directed transport to the target site, uptake in and/or distribution inside a target cell.

6. The oligonucleotide according to claim 5, characterized in that the nucleic acid construct is an antisense oligonucleotide, a DNAzyme, a peptide nucleic acid, a ribozyme and/or an siRNA.

7. The oligonucleotide according to claim 6, characterized in that the antisense oligonucleotide is a phosphothioate antisense oligonucleotide.

8. The oligonucleotide according to claim 1, characterized in that the oligonucleotides comprise the sequences ggaccaccgcaucucuacadtdt, uguagagaugcggugguccdtdt, agcauucguccgguugcgcdtdt, gcgcaaccggacgaaugcudtdt, gaggtggcggcggcggcatgggtgccccgacgttgcc, cccactgagaacgagccagacttggcccagtgtttc, gacgaccccatagaggaacataaaaagcattcgtccggttgcgct, cccagagtggctgcaccacttccagggtttattc, cctgttttgtcttgaaagtggcaccagaggtgc, tttttgggggctcatttttgctgttttgattcccgggcttaccaggt, ttcacagaatagcacaaactacaattaaaactaagcacaaagccatt, cgtctggcagatactccttttgccactgct, aaaggcagtggcctaaatccctttttaaatgacttggctcgatgctgt, cgcagtccgcccaggtccccgctttctttggag, catgccgccgccgccacctc, ggggcacccatgccgccgcc, gcaacgtcggggcacccatg, atgttcctctatggggtcgt, tttatgttcctctatggggt, ccggacgaatgctttttatg, gcaaccggacgaatgctttt, aagcgcaaccggacgaatgc, tggtgcagccactctgggac, aagtggtgcagccactctgg, aataaaccctggaagtggtg, gggaataaaccctggaagtg, ggcaccagggaataaaccct, gctggtggcaccagggaata, caaaaatgagcccccaaaaa, cagcaaaaatgagcccccaa, caaaacagcaaaaatgagcc, tggtaagcccgggaatcaaa, acctggtaagcccgggaatc, tggctttgtgcttagtttta, aatggctttgtgcttagttt, ggatttaggccactgccttt, aaggatttaggccactgcct, caagtcataaaaaggatt, gcatcgagccaagtcata, agcatcgagccaagtcat, cagtctcagtactgaagctg, cagcttcagtactgagactg, ggacgggtccgcgaggcacg, gcccgcggtgctaatgctc, FAM-gcccgcggtgctaatgctc and/or complementary sequences thereof.

9. A pharmaceutical composition comprising an oligonucleotide according to claim 1, optionally in combination with a pharmaceutically tolerable carrier.

10. A kit comprising an oligonucleotide according to claim 1 and/or a pharmaceutical composition comprising an oligonucleotide optionally in combination with a pharmaceutically tolerable carrier.

11. An array comprising an oligonucleotide according to claim 1 and/or a pharmaceutical composition comprising an oligonucleotide optionally in combination with a pharmaceutically tolerable carrier.

12. A method for diagnosis, prophylaxis, reduction, therapy, follow-up and/or aftercare of diseases associated with cell growth, differentiation and/or division comprising the step of using one chosen from the group consisting of (1) an oligonucleotide being directed against a surviving gene characterized in that the oligonucleotide undergoes specific interaction with a survivin mRNA in a target sequence region selected from the group comprising the target sequence regions 33-51, 41-60, 49-68, 92-114, 261-280, 264-283, 278-297, 282-301, 283-385, 286-305, 501-520, 504-523, 516-535, 519-538, 526-545, 532-551, 716-735, 719-738, 724-743, 740-759, 743-762, 1126-1145, 1128-1147, 1302-1321, 1304-1323, 1317-1336, 1325-1344 and/or 1327-1346, (2) a pharmaceutical composition comprising said oligonucleotide, optionally in combination with a pharmaceutically tolerable carrier, (3) a kit comprising said oligonucleotide and/or said pharmaceutical composition and (4) an array comprising said oligonucleotide and/or said pharmaceutical composition.

13. The method according to claim 12, characterized in that the disease is a tumor disease.

14. The method according to claim 13, characterized in that the tumor is a solid tumor or a leukemia.

15. The method according to claim 14, characterized in that the solid tumor is a tumor of the urogenital tract and/or gastrointestinal tract.

16. The method according to claim 13, characterized in that the tumor is a colon carcinoma, mammary carcinoma, stomach carcinoma, pancreas carcinoma, large bowel carcinoma, small intestine carcinoma, ovarian carcinoma, cervical carcinoma, lung carcinoma, prostate carcinoma, bladder carcinoma, large-cell non-Hodgkin lymphoma, melanoma, neuroblastoma, renal cell carcinoma, liver carcinoma, a brain tumor, head-throat tumor, a sarcoma and/or a metastase of the above tumors.

17. The method according to claim 13, characterized in that the solid tumor is a mammary, bronchial, colorectal, prostate carcinoma and/or a metastase of the above tumors.

18. The method according to claim 15, characterized in that the tumor of the urogenital tract is a bladder carcinoma and/or a metastase of said tumor.

19. The method according to claim 12, characterized in that the follow-up is monitoring the course of a disease and/or the effectiveness of an anti-tumor treatment.

20. The method according to claim 12, characterized in that the oligonucleotide is used in a combination therapy, a local and/or systemic therapy.

21. The method according to claim 20, characterized in that the combination therapy comprises a chemotherapy, a treatment with cytostatic agents and/or a radiotherapy.

22. The method according to claim 12 to increase the sensitivity of tumor cells to chemotherapy, radiotherapy or treatment with cytostatic agents.

23. The method according to claim 20, characterized in that the combination therapy comprises a biologically specified form of therapy.

24. The method according to claim 20, characterized in that the combination therapy comprises a gene therapy and/or a therapy using an oligonucleotide for the same or other target molecule.

25. The method according to claim 24, characterized in that said form of therapy is an immune therapy.

26. Array according to claim 11 for inhibiting the viability, the proliferation rate of cells in order to induce apoptosis and/or cell cycle arrest.
